(19) United States
(12) Patent Application Publication
(10) Pub. No.: US 2010/0179575 A1
(43) Pub. Date: Jul. 15, 2010

(54) Adjustable sacral mesh fixation device and method

(76) Inventors: Walter Von Pechmann, Bethesda, MD (US); Samuel C. Yoon, Clarksville, MD (US); Keith Lipford, Baltimore, MD (US); Brian Lipford, Bel Air, MD (US); Austin Cox, Silver Spring, MD (US)

Correspondence Address: OBER / KALER;C/O ROYAL W. CRAIG, 120 EAST BALTIMORE STREET, SUITE 800, BALTIMORE, MD, 21202, US

(21) Appl. No.: 12/590,594

(22) Filed: Nov. 10, 2009

Related U.S. Application Data
(60) Provisional application No. 61/198,791, filed on Nov. 10, 2008.
(60) Provisional application No. 61/201,795, filed on Dec. 15, 2008.

(51) Int. Cl. A61B 17/00

(52) U.S. Cl. 606/151

(57) ABSTRACT

A sacral mesh fixation device and method of use for sacral colpopexy. The sacral mesh fixation device comprises multiple hinged sections pivoted together at opposite ends and adapted to clamp one or more strips of sacral mesh there between. A latching mechanism is provided for releasably latching the opposing sections shut, thereby releasably clamping the sacral mesh to allow verification of tension. A sequential latching mechanism is provided to fixedly lock the fixation device to the sacral mesh by latching the remaining section to the prior sections. At least one of the sections may be sutured to tissue and the sacral mesh inserted between the sections and releasably clamped in place, thereby releasably affixing the mesh to the tissue or fixedly locking the mesh to the device thereby permanently locking the mesh to the tissue. To use the device, a single mesh fixation device or a plurality of mesh fixation devices may be introduced through a trocar into the abdomen, positioned in a single or supporting array, and sutured to surrounding tissue. The sacral mesh is then clamped between opposing sections of the mesh fixation device(s), thereby temporarily suspending the mesh in a sling. However, the fixation devices can be reopened to facilitate easy readjustment and repositioning of the mesh sling to achieve the proper tension and position or fixedly locked for permanent support of the mesh.

### CROSS-REFERENCE TO RELATED APPLICATION(S)

The present application derives priority from U.S. provisional application Nos. 61/198,791 filed 10 Nov. 2008 and 61/201,795 filed 15 Dec. 2008.

### BACKGROUND OF THE INVENTION

1. Field of the Invention

The present invention relates to medical methods and devices for performing sacral colpopexy.

2. Description of the Background The sacral colpopexy operation is designed to recreate support to the upper vagina by attaching straps of permanent synthetic mesh (typically polypropylene mesh available from Ethicon, Inc. and others) to the upper anterior and posterior vaginal walls and then suspending the other end of the mesh on the anterior surface of the sacrum. This operation is one of many operations described for the correction of pelvic organ prolapse but is considered the gold standard for correction of prolapse of the upper vagina. See, “Surgical management of pelvic organ prolapse in women”, Maher C—Cochrane Database Syst Rev—1 Jan. 2007(3):CD004014. This operation can be done either for correction of vaginal vault prolapse in patients who have previously undergone hysterectomy or can be done at the time of hysterectomy in patients with uterine prolapse.

The sacral colpopexy operation was first described as being done through a large incision in the abdominal wall (laparotomy) and is still predominantly done in that manner. FIG. 1 is a diagrammatic illustration of the end result of this surgery, which is usually performed under general anesthesia. An incision is made in the lower abdomen. The bladder and rectum are freed from the vagina and permanent mesh is used to re-suspend the prolapsed vagina, with the mesh secured to the anterior longitudinal ligament of the sacrum (upper tailbone) at one end and attached to the upper front and back vaginal walls at the other end. The peritoneum (lining of the abdominal cavity) is closed over the mesh.

There is growing interest in performing this operation via less invasive approaches, such as laparoscopy or robot-assisted laparoscopic surgery, but this renders the operation more complex and requires a detailed knowledge of anatomy as well as high level of laparoscopic skill. One of the problematic areas in performing laparoscopic or robotic sacral colpopexy is attachment of mesh to the anterior longitudinal ligament of the sacrum. The object is to create a tension-free suspension between the sacrum and the vaginal apex. However, guiding the mesh straps into proper orientation is awkward. Maintaining them in the proper position during suturing requires constant vigilance as they frequently require repositioning. Additionally, maintaining the mesh straps in position occupies one or more instruments that could be utilized elsewhere (for instance in retracting the surrounding tissues for better visualization). Sometimes portions of the mesh will obscure visibility of the presacral surface. Once positioned and sutured in place, significant problems can arise if the mesh is improperly placed. If the mesh is too loosely hung it may be ineffective in supporting the prolapsed vagina. If the mesh is tensioned too tightly, it can lead to urinary incontinence or pain. Because a tension-free yet properly positioned suspension is an important part of the procedure, surgeons will often seek to adjust the tension of the sacral colpopexy mesh straps. Once sutured in place, any adjustment entails removing and then replacing sutures, which is exceedingly difficult especially in the close confines of a laparoscopic procedure. It would be greatly advantageous to facilitate mesh strap adjustment even after suture fixation.

United States Patent Application 20060015001 to Staskin et al. (American Medical) issued Jan. 19, 2006 shows a sling delivery system to treat urological disorders. The U-shaped configuration of the sling assembly also allows the sling to be adjusted during and/or after implantation. However, this device is designed for treatment of incontinence and neither it nor any of the foregoing devices are suitable for performance of sacral colpopexy.

United States Patent Application 20030195386 to Thierfelder et al. (AMS Research Corporation) issued Oct. 16, 2003 shows a surgical kit useful for performing a surgical procedure such as a sacral colpopexy with an implantable Y-shaped suspension for treating pelvic floor disorders such as vaginal vault prolapse. Means are suggested for adjusting the tension using a ratchet wheel, pawl and spring assembly to tighten. However, again there is no way to adjust the suspension after fixation.

United States Patent Application 20080039678 by Montpetit et al. published Feb. 14, 2008 suggests an adjustable sling with four supports the length of which can be increased or decreased. This requires a custom mesh device, whereas a means of allowing adjustment of commercially available mesh would be more advantageous.

It would be greatly advantageous to provide a sacral mesh fixation device and method that facilitates the attachment of mesh to the sacrum and that more readily allows adjustment of standard sacral mesh during and after fixation. This would result in a more efficient, forgiving and easier sacral colpopexy procedure. If the operation can be rendered more efficient, i.e., less time consuming, and with a lower learning curve, there is potential for the operation to be transformed in to one that is done primarily laparoscopically.

### SUMMARY OF THE INVENTION

It is an object of the present invention to provide a sacral mesh fixation device and method for sacral colpopexy that facilitates attachment and adjustment of supporting mesh straps to the sacrum.

Other objects, features, and advantages of the present invention will become more apparent from the following detailed description of the preferred embodiments and certain modifications thereof in which a sacral mesh fixation device and method is provided for sacral colpopexy is disclosed along with its method for use for sacral colpopexy.

The sacral mesh fixation device generally comprises three hinged sections that provide interlockability for clamping to the mesh, as well as anchoring (suturing) one or more of the sections to the anterior longitudinal ligament. The fixation device includes a unidirectional barb system that temporarily stabilizes the mesh in position, as well as providing a latching mechanism for releasably latching the two larger and similarly sized sections of said mesh fixation device together, thereby releasably clamping the sacral mesh in the desired position. One or more sections are suturable to tissue such that sacral mesh can then be inserted between two of the sections and releasably clamped in place, thereby releasably affixing the mesh to the sacrum.

To use the device during a colpopexy procedure, two sutures attached at one end to the middle section of the mesh fixation device and with attached suturing needles at the other end are introduced through a laparoscopic port into the abdomen, sutured through the anterior longitudinal ligament, and then retrieved and removed through the same laparoscopic port. The suture needles are then fed independently through passages in the mesh fixation device, which may or may not be unidirectional. The passages can be simple clearance holes for the suture and needle, or channels with unidirectional barbs or another mechanism that only allows unidirectional suture passage, thereby allowing the suture to be pulled through the channels in a tightening direction but not a loosening direction. The sacral mesh fixation device is then introduced through the same laparoscopic port in to the abdomen. The portions of the suture arms already passed through said channels are then pulled up extracorporeally to create a pulley effect that snugs the sacral mesh fixation device down to the anterior longitudinal ligament. Because of the unidirectional nature of the suture passage channels, the sacral mesh fixation device is fixed in place against the anterior longitudinal ligament, eliminating the need for tying of the suture to achieve fixation of the sacral mesh fixation device to the sacrum. The sutures could then be cut above the sacral mesh fixation device, or if desired, tied together and then cut.

A plurality of such fixation devices can be installed in like manner to support mesh straps. The mesh is inserted between the opposing sections of each mesh fixation device, and the mesh is then automatically suspended on unidirectional barbs projecting away from one section of the sacral mesh fixation device toward the other section, such that the mesh can be easily pulled tighter but not looser. To loosen tension, the mesh straps would need to be removed from between the two sections and then reintroduced between them at less tension. Projecting from the surface of one of the two sections to sit between the two sections is a flexible tongue shaped probe that allows easy feeding of the mesh straps into position, simultaneously pushing the mesh against the projecting unidirectional barbs to prevent loosening of tension. Once the two mesh straps appear to be in the desired position, the more superficial of the two device sections is hinged toward the section that is fixed to the sacrum, resulting in adjustably locking in place of the more superficial section via a reversible locking mechanism, thereby clamping the mesh in the desired position. If after inspecting the elevation of the vagina and determining that repositioning of the mesh is desired, the reversible clamp mechanism is opened, the mesh is retensioned, and the process is repeated to lock the mesh in place. Once the desired tension is confirmed, with the reversible clamp mechanism in the closed position, a second clamp mechanism on the third section is closed irreversibly to fully lock the sections together and stabilize the mesh in the desired position. The hinge on the third section is designed such that cutting of the hinge would be possible to release the irreversible clamp mechanism.

In this manner the mesh can be easily repositioned as desired and then reliably anchored to the anterior longitudinal ligament.

### BRIEF DESCRIPTION OF THE DRAWINGS

Other objects, features, and advantages of the present invention will become more apparent from the following detailed description of the preferred embodiment and certain modifications thereof, in which

FIG. 1 is a diagrammatic illustration of the completed sacral colpopexy surgery in which straps of mesh attached to the upper vagina inferiorly are suspended on the anterior longitudinal ligament of the sacrum superiorly.

FIG. 2 is a top side perspective view of a sacral mesh fixation device according to one embodiment of the invention.

FIG. 3 is a bottom side perspective view of the sacral mesh fixation device as in FIG. 1.

FIG. 4 is a top view of the sacral mesh fixation device as in FIGS. 1-3.

FIG. 5 is a bottom view of the sacral mesh fixation device as in FIGS. 1-4.

FIG. 6 is a rear view of the sacral mesh fixation device as in FIGS. 1-5.

FIG. 7 is a front view of the sacral mesh fixation device as in FIGS. 1-6.

FIG. 8 is a side view of the sacral mesh fixation device as in FIGS. 1-7.

FIGS. 9-19 are side perspective views representing the successive steps involved in the method of sacral mesh fixation to the sacrum.

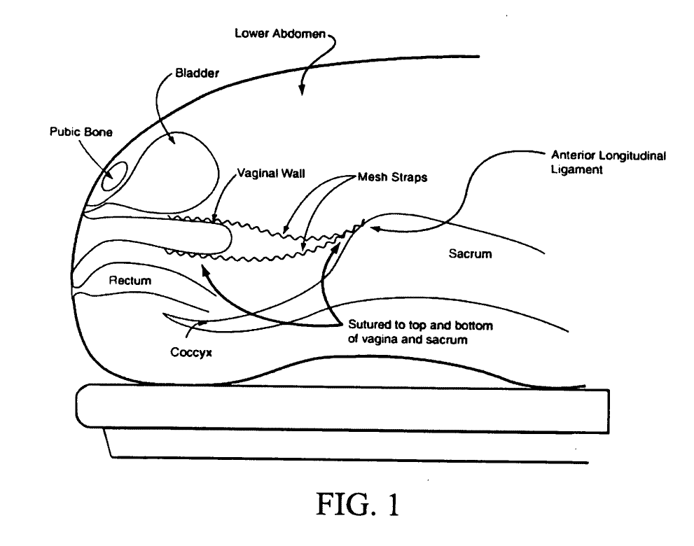

### DETAILED DESCRIPTION OF THE PREFERRED EMBODIMENTS

As described above, the present invention is a mesh fixation system to the sacrum that is part of a sacral colpopexy, and a method of using the same that renders attachment of supporting mesh straps less time consuming, less prone to error, and more susceptible to laparoscopic delivery.

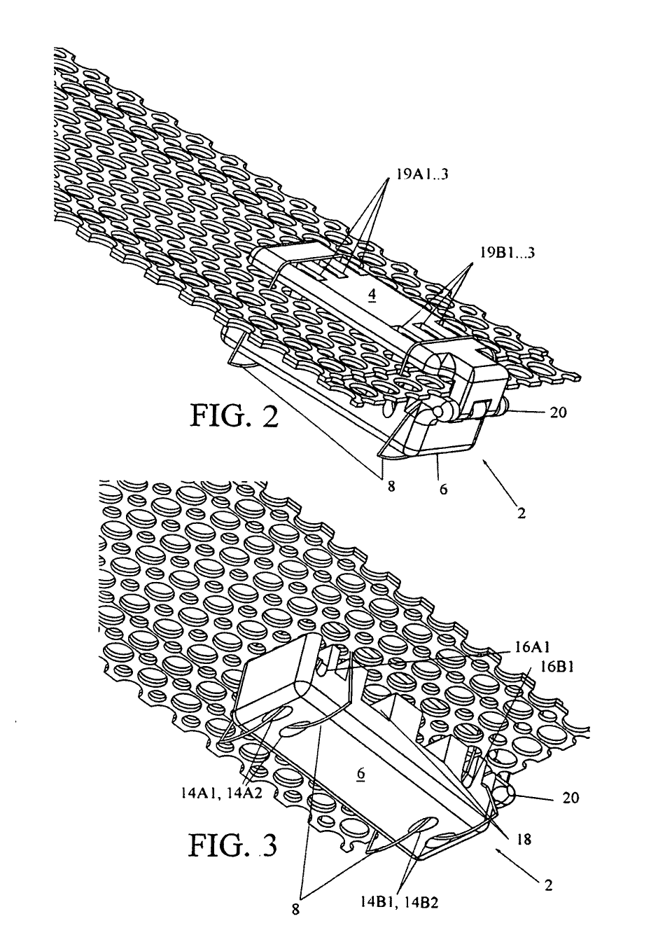

FIG. 2 is a top side perspective view of a sacral mesh fixation device 2 according to one embodiment of the invention, and FIG. 3 is a bottom side perspective view. The sacral mesh fixation device 2 generally comprises three hinged sections: a base section 6; an intermediate latching section 4; and a final latching section 1, all of which provide interlockability for clamping one or more mesh straps 25 to the fixation device as well as enabling a means of anchoring (suturing) one or more of the sections of the mesh fixation device to the anterior longitudinal ligament of the sacrum.

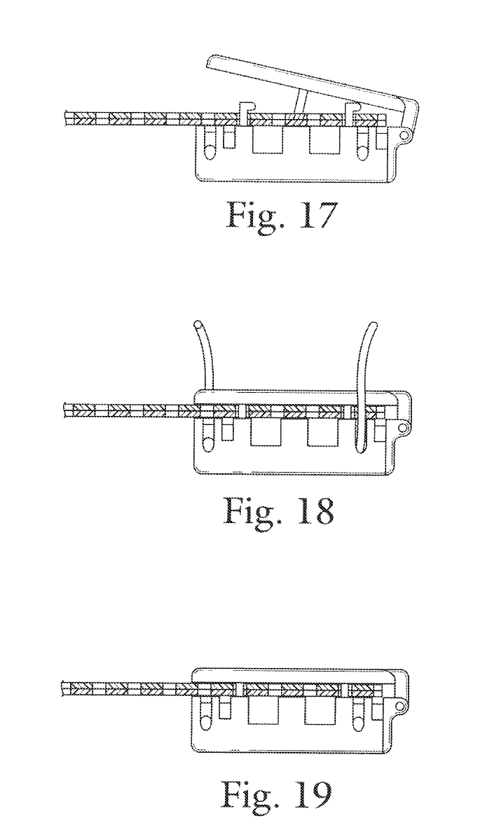

In general use, the base section 6 of the sacral mesh fixation device 2 is sutured to tissue in a desired position by passing two loops of suture 8 through holes 14 in section 6 as shown in FIGS. 2 and 3. Then, with intermediate and final latching sections, 4 and 1, respectively, pivoted open relative to base section 6 via hinges 20 and 7, respectively, one or more sheets of mesh 25 may be inserted between the sections as shown in FIG. 14. Intermediate latch 4 is then releasably latched onto base 6, as shown in FIG. 17, to secure one or more sacral mesh straps 25 therein. Once secured with this intermediate latch 4, the surgeon can inspect the tension and position of the mesh strap(s) to confirm that the placement is acceptable or if one or more of the mesh straps 25 need to be readjusted. If readjustment is needed, this can be easily accomplished by reopening intermediate latch 4 from section 6 and adjusting the mesh 25 until acceptable. When the surgeon is ultimately satisfied with the position of the mesh strap(s) 25, intermediate latch 4 is latched (snapped) onto base section 6, and then final latch 1 is snapped over intermediate latch 4 and base section 6. Once the final latch 1 is snapped in place, the fixation device is fixedly locked together and can no longer be adjusted. At this point, if the fixation device 2 is desired to be moved or readjusted, the surgeon will need to destructively sever (cut) final latch 1 (to reopen latches 1 and 4) and sever sutures 8 to fully remove the device 2. This will require the removal of the device 2, replacement with a new device 2, reattachment of new sutures and reinstallation of the mesh 25.

FIG. 11 is a top side perspective view of the sacral mesh fixation device 2 illustrating an exemplary latching mechanism. The base 6 is formed with a series of rectangular slots 32 aligned across an end as shown in FIG. 5. Base 6 may also be formed with an overhanging resilient feed tongue 9 for adjustably securing the mesh strap there under until the desired amount of tension is achieved. The intermediate latch 4 is formed with a series of protrusions 3, as shown in FIG. 11, which are aligned such that they fit into the slots 32 in base 6. The protrusions are intended to contact the mesh 25 when intermediate latch 4 is closed against base 6, pushing mesh 25 down through the slots 32 in base 6. This is intended to have two functions: 1) to capture and hold the mesh 25 between the protrusions 3 and the mating slots 32 in base 6; and 2) to force the mesh to displace towards the underside of base 6 such that the mesh 25 makes contact with the anterior longitudinal ligament of the sacrum. This latter function will, in time, cause the mesh 25 to naturally adhere to adjacent tissue around the anterior longitudinal ligament and provide additional anchoring of the device to the anterior longitudinal ligament. When the intermediate latching section 4 is closed the sacral mesh is temporarily locked therein.

Closure of the final latching section 1 over the intermediate latching section 4 makes the fixture semi-permanent.

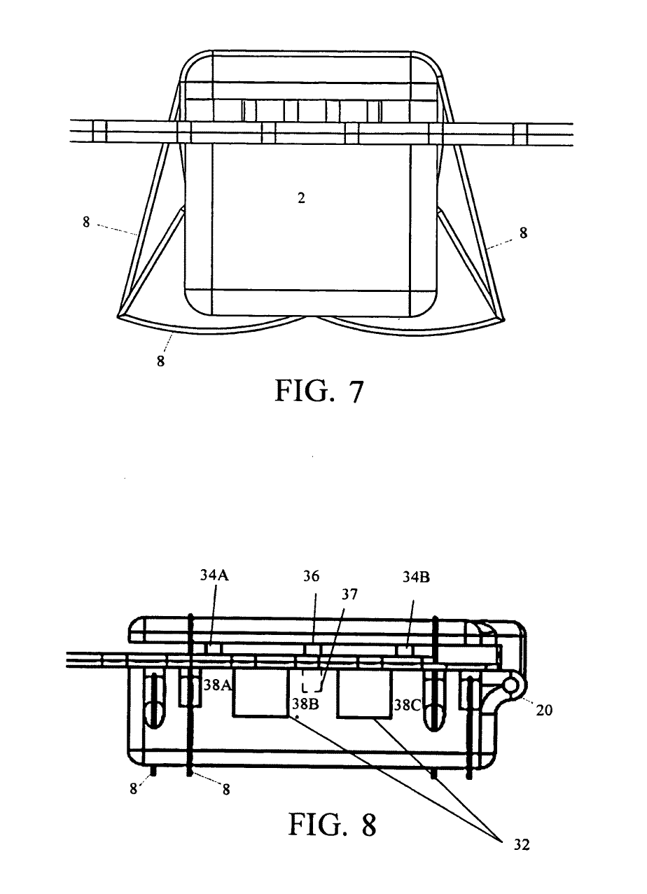

FIG. 5 also illustrates the location of the sutures 8. The base 6 is sutured to the anterior longitudinal ligament by passing two loops of suture 8 through two respective suture holes 14, and through the anterior longitudinal ligament. This does not interfere with opening or closing of the device so the mesh 25 can still be adjusted. FIG. 6 is a rear view of the sacral mesh fixation device 2 showing suture loops 8 on both sides of the device 2. FIG. 7 is a front view of the sacral mesh fixation device 2 which shows that the suture loops 8 at the other end are substantially symmetric to those of FIG. 6.

FIG. 8 is a side view of the sacral mesh fixation device 2 in its fixedly locked position. Each latch 4 and 1, is formed with cooperating hinges 20 and 7 at opposing ends, respectively. The hinges on each latch 4 and 1 may be pin-in-hole type as shown, or a pin-together configuration with one section comprising an axle with opposing hubs and the other comprising pin-in mating features for receiving the axle and allowing pivoting. Alternatively the hinges can be one-piece, molded, plastic pin-less hinges (made from the same contiguous material as the latch, albeit thinner, that stretches and bends as needed in order to function as a hinge). One skilled in the art will readily understand that a variety of pin-in or other hinge configurations will suffice.

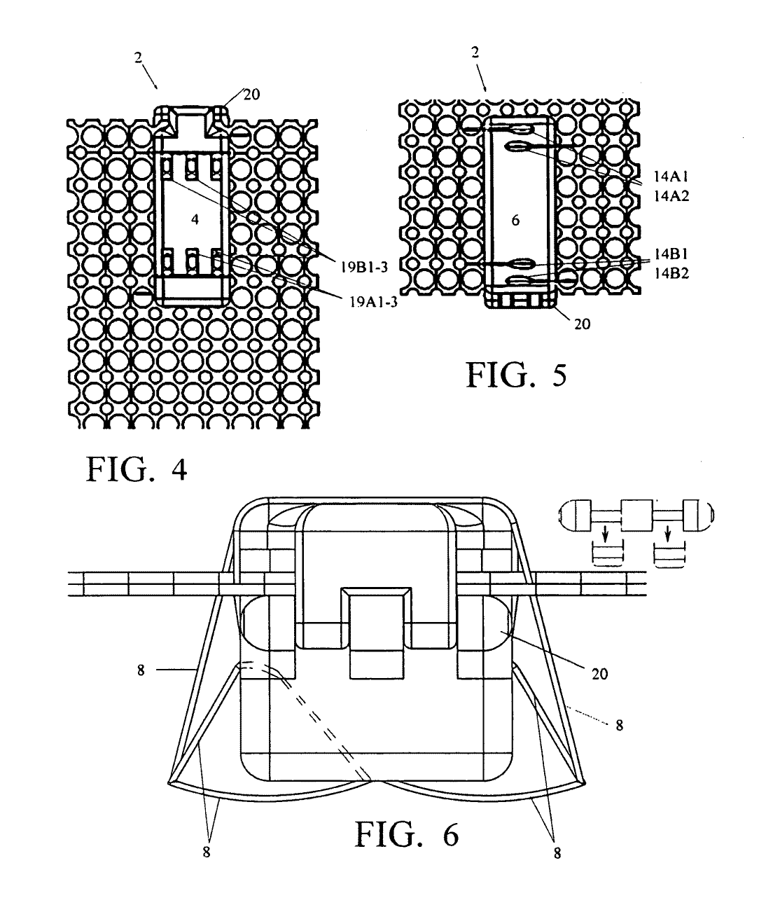

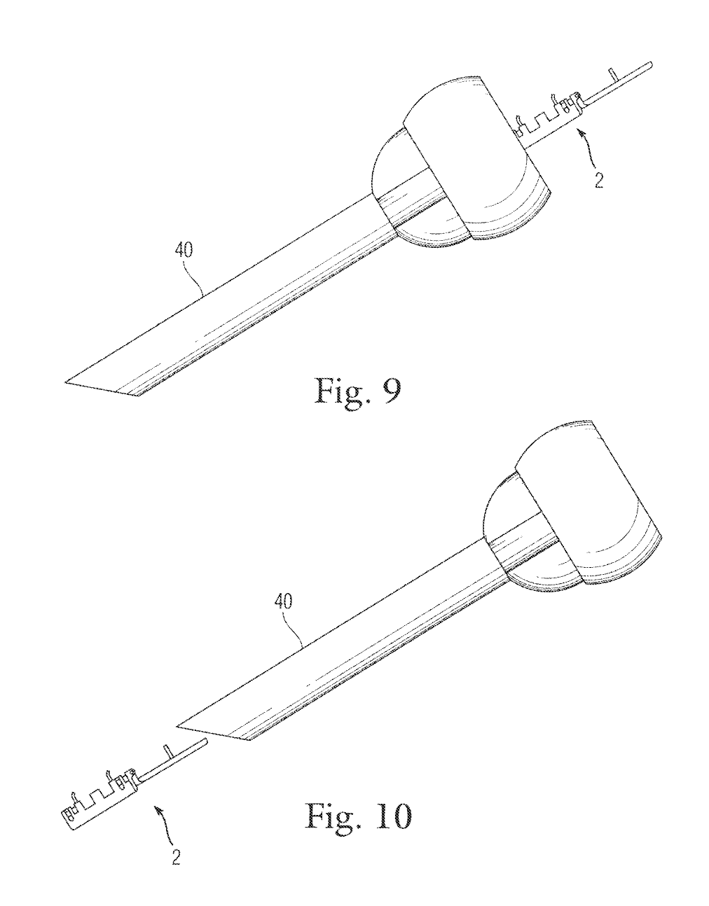

FIGS. 9-19 are side perspective views representing the successive steps involved in the method of sacral mesh fixation using the device of FIGS. 2-8.

As shown at FIGS. 9-19, the mesh fixation device 2 is introduced in an open configuration by an introducer (not shown) through a trocar 40 inserted in a laparoscopic port into the abdomen.

As seen in FIG. 11, suturing needles 21 are used to run two suture anchor loops 8 down holes 14a in base section 6, through the sacral ligament, and back up holes 14b. Holes 14b could be simple clearance holes for the suture and needle, or may be unidirectional suture channels which only allow the suture to be pulled through the channels in one tightening direction but not a loosening direction, thereby eliminating the need to secure the suture with knots.

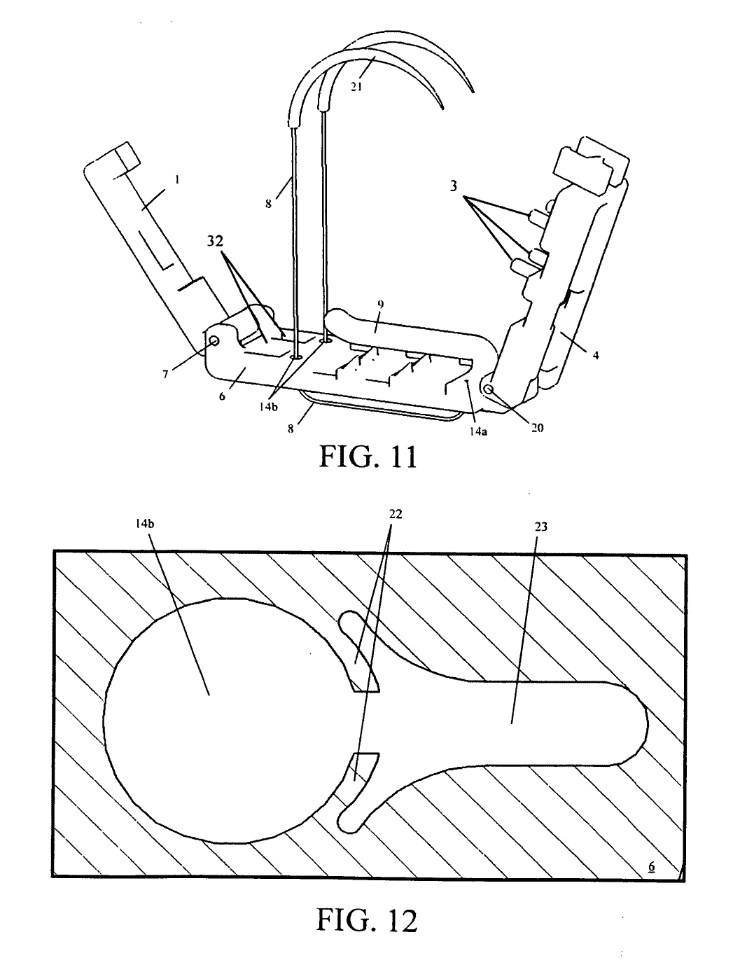

FIG. 12 illustrates one possible implementation of unidirectional suture holes 14b adapted with offset channel locks 23 which allow the needle/suture to pass through suture hole 14b until the surgeon purposefully chooses to lock the suture in place by pulling the suture transversely into the channel lock 23. Protruding flexible fingers 22 prevent the suture from accidentally entering locking section 23 while the surgeon is tightening the suture. Employing unidirectional suture channel locks 23 are effective yet optional features. The suture may be prevented from re-entering suture holes 14b during the lifetime of the sacral mesh fixation device 2 by mating features in the intermediate latch 4 that fill clearance holes 14b during later locking steps.

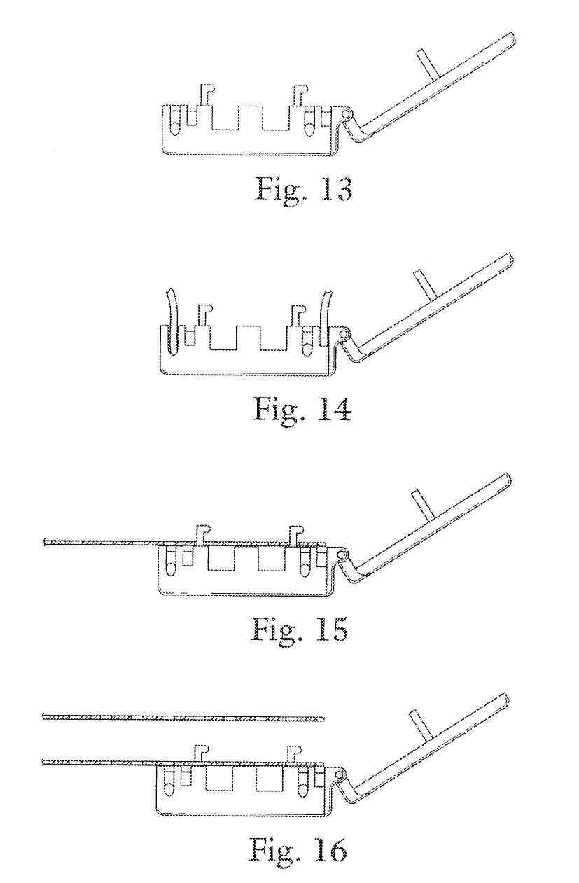

At this point, as shown in FIG. 13, the mesh fixation device 2 is positioned with the floor of the base 6 lying against the anterior longitudinal ligament, (still in an open position). As seen in FIGS. 14 and 11, two suture anchor loops 8 are applied to attach the mesh fixation device 2 to the anterior longitudinal ligament. Both suture loops 8 fix the fixation devices 2 to the anterior longitudinal ligament but do not obstruct the clamping or releasing action of the mesh fixation device 2.

Since the mesh 25 straps are not yet applied, there is no difficulty as normally associated with maintaining the mesh 25 straps in position or in manipulating the straps or surrounding tissue for better visualization.

Once the fixation device 2 is placed, as seen in FIGS. 15-16, the ends of each strap of mesh 25 to be fixed are placed into the open device 2 (as part of a normal sacral colpopexy procedure, after the straps are affixed to the vaginal muscularis). Once placed into the device, each mesh strap is pulled sequentially under the feed tongue 9 in the direction of the angled barbs 10 (see FIGS. 11 and 16) until the desired amount of tension is achieved. The end of each mesh 25 strap is then successively released against the angled barbs 10 such that the mesh 25 is held in place.

As shown in FIG. 17 and described in more detail above, the intermediate latch 4 is then closed onto base 6 such that the lock feature 11 on intermediate latch 4 releasably locks onto base 6. The current invention uses a snap fit for the lock feature between the mating parts, but one skilled in the art will readily understand that a variety of lock design configurations could suffice. If the tension and position of the mesh straps 25 is determined to be incorrect, a latch release 12 on intermediate latch 4 is squeezed together using forceps (or other laparoscopic tools) causing the lock feature 11 to release from base 6, which enables opening of intermediate latch 4 from base 6 and subsequent retentioning of the mesh 25 straps. Once mesh 25 strap tension is verified, intermediate latch 4 and final latch 1 are sequentially closed onto base 6. Final latch 1 has a similar snap lock feature 13 that fixedly locks final latch 1 onto a mating feature on base 6 (as shown in FIG. 15), which permanently locks the device 2 onto the mesh 25 straps to prevent accidental release (as shown in FIG. 18).

The preferred embodiment for the current invention employs a suitable bio-compatible material such as thermo-plastic (injection moldable) for all parts of the device 2. For example, Peek-Optima is a suitable implantable biomaterial for the surgical and medical device markets, sold by Invibio® Biomaterial Solutions. One skilled in the art will readily understand that a variety of materials could suffice, including stainless steel.

One skilled in the art should readily understand that the above-described mesh fixation device 2 and method of using them for sacral colpopexy greatly facilitates the initial attachment of supporting mesh 25 straps because it is much easier to suture the fixation device 2 rather than the unwieldy mesh 25 itself, and the fixation device 2 then facilitates easy readjustment of the mesh 25 strap to achieve the proper tension and position.

Having now fully set forth the preferred embodiment and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

We claim:

1. A sacral mesh fixation device for sacral colpopexy, comprising:
a mesh fixation device including a base and two opposing sections pivoted together at opposite ends and adapted to clamp sacral mesh there between, one of said sections being suturable to tissue; and
a latching mechanism for releasably latching the sections of said mesh fixation device together, thereby releasably affixing them to the device; and
a sequential latching mechanism for fixedly latching said mesh fixation device together to to permanently lock said sacral mesh to said tissue.

2. A method for attaching sacral mesh during a colpopexy procedure to the sacrum, comprising the steps of:
introducing a mesh fixation device through a trocar into the abdomen, said mesh fixation device comprising a base and two opposing sections pivoted together at opposite ends;
positioning the mesh fixation device with one or more of said sections sutured against a tissue, and the other sections pivoted to an open position;
suturing one or more said sections to said tissue;
inserting one or more sacral meshes between the opposing sections of the mesh fixation device;
adjustably tensioning each said mesh within the fixation device
adjustably locking the device around each said mesh
fixedly locking said opposing sections over the sacral mesh and latching them together to affix the mesh there between.

3. The method according to claim 2, wherein the device is fabricated from a bio-compatible, thermo-plastic (injection moldable) material.

4. The method according to claim 2, wherein said device exposes the affixed mesh into contact with the anterior longitudinal ligament of the sacrum, allowing said mesh 25 to, over time, naturally adhere to adjacent tissue around the anterior longitudinal ligament and provide additional anchoring of the device to the anterior longitudinal ligament.

5. A sacral mesh fixation device for securing a sacral mesh to tissue in a sacral colpopexy procedure, comprising:
a first section adapted for suturing to said tissue;
a second section pivoted at one end to an end of said first section and adapted to releasably clamp said sacral mesh there between.

6. The method according to claim 5, further comprising a third section pivoted at one end to an opposing end of said first section and adapted to permanently clamp said sacral mesh there between.
